(19) United States
(12) Patent Application Publication
(10) Pub. No.: US 2024/0277416 A1
(43) Pub. Date: Aug. 22, 2024

(54) SYSTEM AND METHOD FOR MULTIMODAL DISPLAY VIA SURGICAL TOOL ASSISTED MODEL FUSION

(71) Applicant: EDDA TECHNOLOGY, INC., PRINCETON, NJ (US)

(72) Inventors: Xiaonan Zang, Princeton, NJ (US); Guo-Qing Wei, Plainsboro, NJ (US); Li FAN, Belle Mead, NJ (US); Xiaolan ZENG, Princeton, NJ (US); Jianzhong QIAN, Princeton Junction, NJ (US)

(21) Appl. No.: 18/172,447

(22) Filed: Feb. 22, 2023

(51) Int. Cl. A61B 34/20; A61B 34/10

(52) U.S. Cl. CPC A61B 34/20; A61B 34/10; A61B 2034/107; A61B 2034/2065

(57) ABSTRACT

The present teaching relates to automated generation of a surgical tool-based visual guide. Two-dimensional (2D) images capturing anatomical structures and a surgical instrument therein are provided during a surgery. The type and pose of a tool attached to the surgical instrument are detected based on the 2D images. Focused information is determined based on the type and pose of the detected tool and is used to generate a visual guide to assist a user to perform a surgical task using the tool.

### CROSS REFERENCE TO RELATED APPLICATION

The present application is related to U.S. patent application Ser. No. ______ (Attorney Docket No. 140551.569672) filed on ______, entitled “SYSTEM AND METHOD FOR SURGICAL TOOL BASED MODEL FUSION”, the contents of which are incorporated herein by reference in its entirety.

### BACKGROUND

### 1. Technical Field

The present teaching generally relates to computers. More specifically, the present teaching relates to signal processing.

### 2. Technical Background

With the advancement of technologies, more and more tasks are now performed with the assistance of computers. Different industries have benefited from such technological advancement, including medical industry, where large volume of image data, capturing anatomical information of a patient, may be processed by computers to identify anatomical structures of interest (e.g., organs, bones, blood vessels, or abnormal nodule), obtain measurements for each object of interest (e.g., dimension of a nodule growing in an organ), and visualize relevant features (e.g., three-dimensional (3D) visualization of an abnormal nodule). Such techniques have enabled healthcare workers (e.g., doctors) to use high-tech means to assist them in treating patients in a more effective manner. Nowadays, many surgeries are performed with laparoscopic guidance, making it often unnecessary to open up the patient, minimizing the damaged to the body.

Via the guidance of laparoscopy, a surgeon may be guided to approach a target organ and perform what is needed. Although each surgery may have a predetermined objective, there may be different subtasks in each surgery to accomplish in order to achieve the predetermined objective. Examples include maneuvering a surgical instrument to a certain location with the guidance of laparoscopic images, aiming at the anatomical structure where some subtask is to be performed, and then execute the subtask using the surgical instrument. Exemplary subtasks include using a surgical tool to separating blood vessels from an organ to be operated on, clapping the blood vessels to stop the blood flow prior to resection a part of an organ, etc. Different surgical instruments or tools may be needed for performing different subtasks.

While laparoscopic images may provide a user live visualization of the anatomies inside a patient's body, there are limitations on how to interpret these images. First, these laparoscopic images are usually two dimensional (2D) so that certain information of the 3D anatomies may not be displayed in the 2D images. Second, as a laparoscopic camera has a limited field of view, the acquired 2D images may capture only a partial view of the targeted organ. Moreover, some essential anatomical structures, such as blood vessels, may reside inside of an organ so that they are not visible in 2D images. Due to these limitations, a user needs to mentally reconstruct what is not visible which requires substantial experience to digest what is seen from laparoscopic images to align what is observable with the preplanned 3D surgical procedure. Thus, there is a need for a solution that addresses the challenges discussed above.

### SUMMARY

The teachings disclosed herein relate to methods, systems, and programming for information management. More particularly, the present teaching relates to methods, systems, and programming related to hash table and storage management using the same.

In one example, a method, implemented on a machine having at least one processor, storage, and a communication platform capable of connecting to a network for automated generation of a surgical tool-based visual guide. Two-dimensional (2D) images capturing anatomical structures and a surgical instrument therein are provided during a surgery. The type and pose of a tool attached to the surgical instrument are detected based on the 2D images. Focused information is determined based on the type and pose of the detected tool and is used to generate a visual guide to assist a user to perform a surgical task using the tool.

In a different example, a system is disclosed for automated generation of a surgical tool-based visual guide, which includes a surgical tool detection unit, a tool-based focused information identifier, and a focused information display unit. The surgical tool detection unit is provided for detecting the type and pose of a tool attached to a surgical instrument based on 2D images that capture anatomical structures and the surgical instrument. The tool-based focused information identifier is provided for determining focused information based on the type and pose of the tool. The focused information display unit is provided for generating a visual guide based on the focused information for assisting a user to perform a surgical task using the tool.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for automated generation of a surgical tool-based visual guide. The information, when read by the machine, causes the machine to perform various steps. Two-dimensional (2D) images capturing anatomical structures and a surgical instrument therein are provided during a surgery. The type and pose of a tool attached to the surgical instrument are detected based on the 2D images. Focused information is determined based on the type and pose of the detected tool and is used to generate a visual guide to assist a user to perform a surgical task using the tool.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A shows a surgical instrument with a hook at one end nearing a target organ;

FIG. 1B shows a surgical instrument with a cutter at one end nearing a target organ;

FIG. 2A depicts an exemplary high level system diagram of a surgical tool assisted model fusion mechanism, in accordance with an embodiment of the present teaching;

FIG. 2B is a flowchart of an exemplary process of a surgical tool assisted model fusion mechanism, in accordance with an embodiment of the present teaching;

FIG. 3A depicts an exemplary high level system diagram of a surgical tool detection unit, in accordance with an embodiment of the present teaching;

FIG. 3B is a flowchart of an exemplary process of a surgical tool detection unit, in accordance with an embodiment of the present teaching;

FIG. 3C shows an exemplary implementation of a surgical tool detection unit, in accordance with an embodiment of the present teaching;

FIG. 4A shows an exemplary surgical tool based focused information configuration, in accordance with an embodiment of the present teaching;

FIG. 4B illustrates an example of different focused information determined because of a different surgical tool detected, in accordance with an embodiment of the present teaching;

FIG. 4C illustrates an example of focused information determined based on a surgical tool detected, in accordance with an embodiment of the present teaching;

FIG. 5A depicts an exemplary high level system diagram of a tool-based focused information identifier, in accordance with an embodiment of the present teaching;

FIG. 5B is a flowchart of an exemplary process of a tool-based focused information identifier, in accordance with an embodiment of the present teaching;

FIG. 5C depicts an exemplary high level system diagram of a 2D/3D focused region identifier, in accordance with an embodiment of the present teaching;

FIG. 5D is a flowchart of an exemplary process of a 2D/3D focused region identifier, in accordance with an embodiment of the present teaching;

FIG. 6 is an exemplary high level system diagram of a focused information display unit, in accordance with an embodiment of the present teaching;

FIG. 7A is a flowchart of an exemplary process of a focused information display unit, in accordance with an embodiment of the present teaching;

FIG. 7B is a flowchart of an exemplary process of a focused overlay renderer for displaying focused information identified based on a tool detected, in accordance with an embodiment of the present teaching;

FIG. 7C illustrates an exemplary display of focused information near a surgical instrument with an enlarged view showing details of the focused information, in accordance with an embodiment of the present teaching;

FIG. 8 is an illustrative diagram of an exemplary mobile device architecture that may be used to realize a specialized system implementing the present teaching in accordance with various embodiments; and

FIG. 9 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well known methods, procedures, components, and/or system have been described at a relatively high-level, without detail, in order to avoid unnecessarily obscuring aspects of the present teachings.

The present teaching discloses exemplary methods, systems, and implementations for automatic determining information of focus on-the-fly based on a surgical tool detected and displaying of such information of focus. A surgical instrument is inserted into a person's body for perform a predetermined surgical operation. The surgical instrument may carry on its tip end a tool to be used for carrying out some tasks. The surgical instrument's position in a 3D workspace is tracked so that its 3D position is known. 3D models for a target organ to be operated on are registered with 2D images acquired during the surgery. In addition to tracking the 3D position of n surgical instrument, the tool attached to the tip of the instrument may also be detected based on the 2D images.

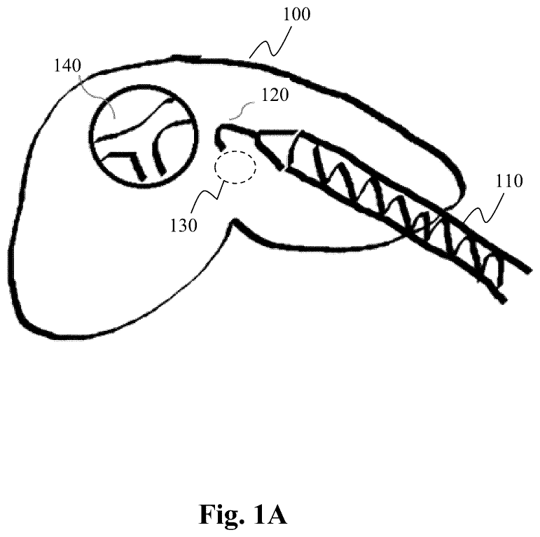

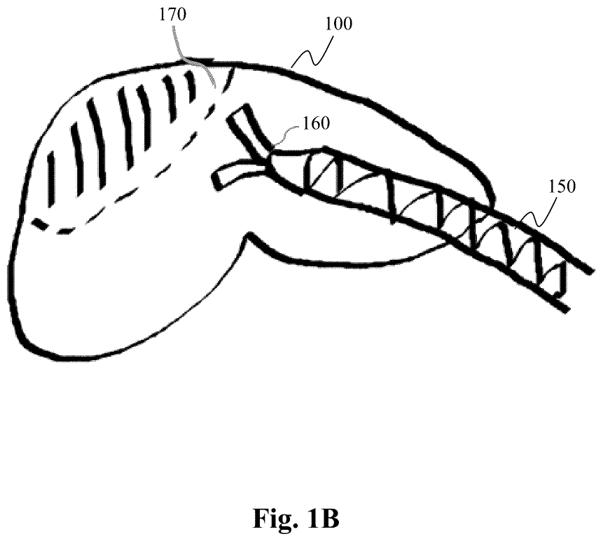

Each tool may have certain designated function(s) to perform during a surgery. As such, the presence of a tool may signal what is the tasks to be carried out, what is the focus of the user (surgeon) at the moment, and what information may be relevant in the sense that it may assist the user to do the job with a greater degree of ease. For instance, a hook 120 may be attached to the tip of a surgical instrument 110 when the surgical instrument appears near a target organ 100, as shown in FIG. 1A. As it is known that a hook is usually used to handle tasks related to blood vessels, detection of the presence of a hook from 2D images may signal that the user intends to do something with the blood vessel related to the target organ. Such tasks may include hooking on a blood vessel so that to separate the vessel from the organ before resecting the organ, stopping the blood flow by clamping the vessel or by burning the vessel. In this situation, it may be inferred that the focus of the user is on vessels. Given that, enhancing the display of vessels that are approximate to the hook may help the user to perform the intended task. For instance, as illustrated in FIG. 1A, if the area 130 is at where the hook 120 is, an enlarged view 140 of the vessels in area 130 may be displayed which provides much better view of the details in the area of interest.

FIG. 1B illustrates another example, where a different tool such as a cutter 160 may be attached to the tip of a surgical instrument 150 and appears near an organ 100. It may be known that such a cutter 160 is usually for resecting an organ. As resection operation generally has a preplanned surgery trajectory, when a cutter is detected in 2D images, it may indicate that the user is ready to resect the organ 100. Given this assessment, the information of focus is the part of the organ that is to be removed. To assist the user to visualize better the boundary for the resection, the focused information may be displayed in an emphasized manner on the part of the organ to be removed, e.g., 170 in FIG. 1B, and the removal trajectory, e.g., the dotted boundary, on organ 100. In this case, the focused information determined based on the detected surgical tool may be displayed in some special way. For instance, the corresponding resection region in the 3D model may be projected onto the 2D image with, e.g., highlights and the boundary of the planned resection trajectory may be displayed in, e.g., some different color so that it is easier for the user to see and to follow. The details of the focused information may also be enlarged either superimposed on a part of the screen or in a pop-up window.

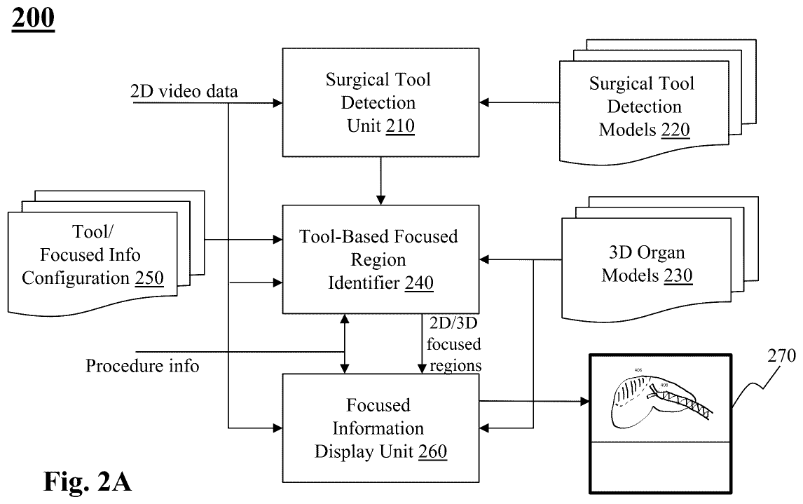

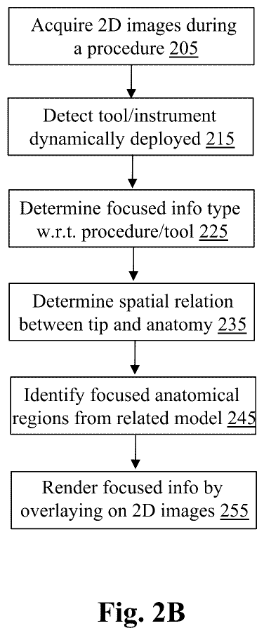

The present teaching as disclosed herein determines what is the focused information that may help a user to accomplish the current task based on a surgical tool currently detected and displays accordingly such identified focused information to the user in a manner that improves the effectiveness of accomplishing the tasks in hand. FIG. 2A depicts an exemplary high level system diagram of a surgical tool assisted model fusion mechanism 200, in accordance with an embodiment of the present teaching. In this illustrated embodiment, the surgical tool based model fusion mechanism 200 comprises a surgical tool detection unit 210, a tool-based focused region identifier 240, and a focused information display unit 260.

2D video images are provided as input to these three components. As discussed herein, in the surgical workspace, calibration may have been performed to facilitate registration of 2D images with 3D coordinates of points in the 3D workspace. In some embodiments, such calibration may be performed via a tracking mechanism (not shown) that tracks a surgical instrument having some tracking device(s) attached thereon on one end of the instrument remaining at outside of the patient's body. Feature points present in the 2D images may be registered with the 3D workspace. A transformation matrix may be derived based on a set of feature points identified from 2D images via the surgical instruments (so that the 3D coordinates of such feature points can be derived) and corresponding feature point on the 3D model of the organ. Based on the transformation matrix, any points on the 3D organ model may be projected onto the 2D images to provide 3D visualization of the organ at where the organ appears in the 2D images. Similarly, any type of information such as blood vessels within the organ may also be projected onto the 2D images.

In the illustrated embodiment in FIG. 2A, the surgical tool detection unit 210 is provided for recognizing a surgical tool as appearing in 2D video images which may be acquired by, e.g., a laparoscopic camera inside of a patient's body. The detection result on the type of surgical tool is provided to the tool-based focused region identifier 240 which then determines, in accordance with tool/focused information configuration stored in 250, the focused information related to the detected surgical tool in order to obtain, from 3D organ models stored in 230, the focused information of the target organ. The tool/focused information configuration 250 is provided to specify information of focus with respect to each surgical tool in different surgical procedures. Such configuration may be provided based on medical practice by, e.g., professionals in the field.

A 3D organ model in storage 230 may be generated offline prior to the surgical procedure based on image data obtained from the patient. Such a 3D model may include different kinds of information. For example. It may include 3D modeling of the organ using a volumetric representation and/or surface representation. It may also include modeling of internal anatomical structures, such as a nodule growing in the organ and blood vessels structures around and within the organ. The modeling of the organ and internal anatomical structures may also be provided with different features and measurements thereof about, e.g., the organ itself, a nodule therein, or different vessels. In addition, a 3D model for a patient's organ may also incorporate information on a preplanned surgery trajectory with planned cut points on the surface of the organ with defined positions. Furthermore, a 3D organ model may also include modeling of some regions nearby the organ. For instance, blood vessels or other anatomical structure such as bones outside of the organ may also be modeled as they are connected to the organ or form a part of a blood supply network connected to the organ so that they may impact how the surgery is to be conducted.

Different types of information included in the 3D organ model may be used to be projected on the 2D video images to assist the user to performing different tasks involved in the surgery. At each moment, depending on the type of surgical tool detected, different piece(s) of information from the 3D model may correspond to focused information. For example, when a surgical hook is detected from 2D images, blood vessels may be deemed as focused task at the moment because a surgical hook may usually be used to handle tasks associated with blood vessels. In this case, the information in the 3D organ model 230 characterizing the blood vessels may be obtained from 230 as the focused information and may be used for special display. Although other types of information from the 3D organ model may also be retrieved and used for projection onto the 2D images (e.g., the organ's 3D shape and measures), the part that is deemed as the focused information may be displayed in a different way. One example is illustrated in an exemplary display 270 in FIG. 2A, where the 3D model for the organ is projected on to 2D images but with a part of the organ to be removed displayed in an, e.g., highlighted manner because a surgical cutter is detected from the 2D images. As shown, in the highlighted area corresponding to a resection region, the boundary information may also be specifically marked in a noticeable way because that is where the surgical cutter needs to approach to make the cut.

FIG. 2B is a flowchart of an exemplary process of the surgical tool assisted model fusion mechanism 200, in accordance with an embodiment of the present teaching. 2D images may be acquired at 205 from, e.g., a laparoscopic camera inserted in a patient's body. From the 2D images, the surgical tool detection unit 210 recognizes, at 215, the type of surgical tool as it is deployed at the moment of the surgery. Such detection result is sent to the tool-based focused region identifier 240, which determines, at 225, the type of focused information corresponding to the detected tool type given the type of surgical procedure based on the tool/focused information configuration 250. As discussed herein, the type of focused information may be identified from a focused region determined based on the location of the surgical tool detected. In some embodiments, the focused region is to be determined with respect to the perspective of the detected surgical tool. To do so, the spatial relationship between a tip of the detected surgical tool and the anatomical part near the tip location is determined at 235. Based on the spatial relationship, the perspective of the surgical tool may be estimated. For example, a 2D region corresponding to a part of the anatomy faced directly with the tip of the tool may include the information relevant to the procedure to be performed using the surgical tool. One example is provided in FIG. 1A, wherein the focused information to be displayed to assist a surgeon to perform an operation using the detected surgical tool corresponds to area 130 because it is an area directly faced by the tip of the surgical hook detected.

Based on the spatial relation between the tool tip and the relevant anatomical part, the tool-based focused region identifier 240 then identifies, at 245, both a 2D focused region in 2D images and a corresponding 3D focused region of appropriate part(s) of the 3D organ model. Such identified 2D/3D focused regions are where the focused information resides and is to be displayed to assist a surgeon to perform an operation using the surgical tool at that moment. Based on the identified 2D/3D focused regions, the focused information display unit 260 then renders, at 255, the content of the 3D model from the 3D focused region by projecting such 3D content onto the 2D focused region in 2D images in a registered manner. In some embodiments, the 3D organ model may be retrieved when the surgery starts and during the surgery relevant parts may then be used for projecting onto the 2D images in a manner determined by the surgical tool detected. For instance, at the beginning of a surgery, the surface representation of a 3D organ in the 3D model may be used to project onto 2D images. During the surgery, the display may be adjusted dynamically. When a surgical instrument changes its pose, e.g., it is oriented towards a different part of the organ, the projection of the surface representation of the 3D model needs to be adjusted so that the part of the surface of the organ directly facing the instrument is displayed via projection.

As another example, when a different surgical tool such as a hook is detected, the blood vessel tree inside the organ may need to be displayed in a way so that the user can “see through” the organ to look at the vessels. In this case, the blood vessel representations of the 3D model may be used for rendering such focused information. In some embodiments, the blood vessels may be projected with an orientation appropriate with respect to the perspective of the tip of the surgical hook, as discussed herein. While the blood vessels are the focus and are rendered in a special way, other parts of the organ may be rendered in such a way to deemphasize so that the focused information is more prominent. Thus, at each moment, depending on the situation, different parts of the 3D model may be used in different ways for different displays. The rendering may consider different rendering treatments of focused and non-focused information. For example, when blood vessels are focal point, other anatomical structures may be displayed to appear faded so that the blood vessels may appear to be more visible. At the same time, the focused information may also be displayed in a highlighted manner to increase the contrast, e.g., increasing the intensity of the pixels on the blood vessel or using a bright color to display blood vessel pixels.

That is, whenever the focus changes, the projection of different pieces of 3D model information onto the 2D images may also change accordingly. Continuing the above example, when later a surgical cutter is detected (after a surgical hook), the blood vessels previously projected in 2D images may now need to be completely hidden and the part of the organ in front of the detected cutter now needs to be displayed in a special and highlighted way so that a part of the organ near the cutter can be visualized clearly. Thus, a 3D organ model, once retrieved from storage 230 may need to be used whenever situation changes and each time, a different part of the 3D model may be used for focused display and other non-focused display.

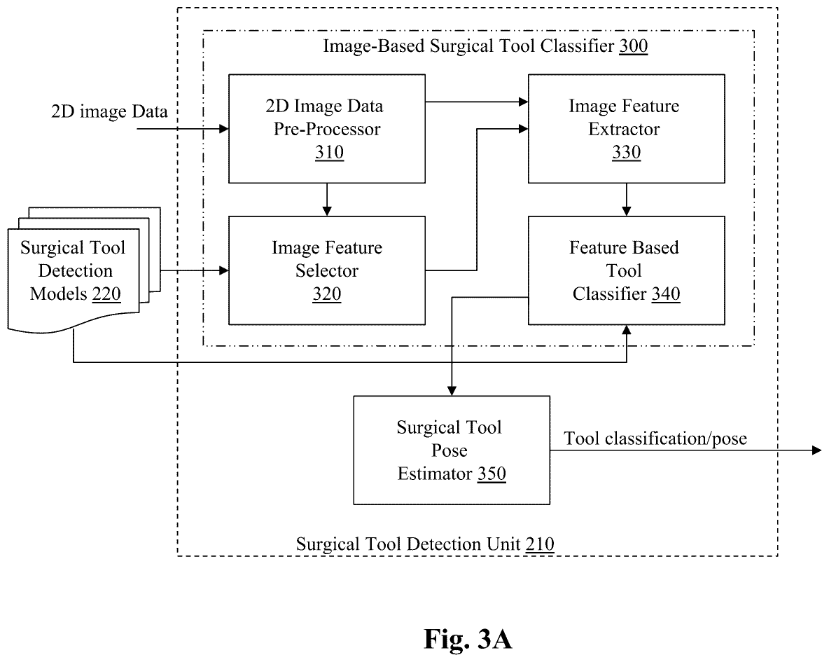

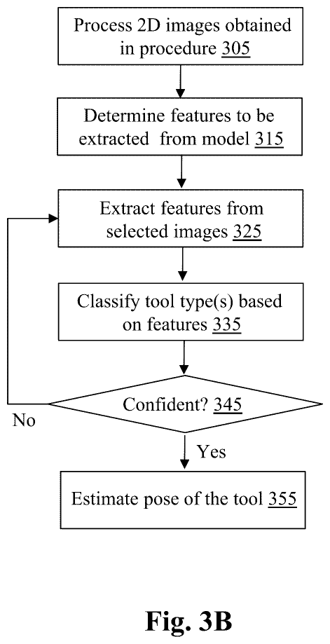

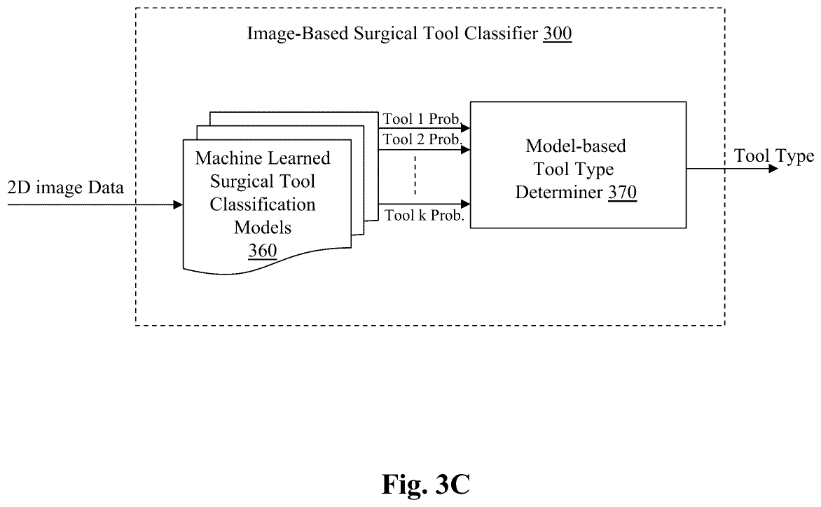

FIG. 3A depicts an exemplary high level system diagram of the surgical tool detection unit 210, in accordance with an embodiment of the present teaching. In this illustrated embodiment, the surgical tool detection unit 210 includes an image-based surgical tool classifier 300 and a surgical tool pose estimator 350. In some embodiments, the image-based surgical tool classifier 300 may be provided as shown in FIG. 3A, and includes a 2D image data pre-processor 310, an image feature selector 320, an image feature extractor 330, a feature-based tool classifier 340, and a surgical tool pose estimator 350. FIG. 3B is a flowchart of an exemplary process of the surgical tool detection unit 210, in accordance with an embodiment of the present teaching. In operation, the 2D image data pre-processor 310 receives 2D images as input and then processes, at 305, the received images. Such preprocessing may be carried out to, enhance the image quality to facilitate feature extraction. Such enhancement may include filtering or intensity enlargement for improving the contrast.

In this illustrated embodiment, to recognize a surgical tool from 2D images, features may be explicitly extracted from preprocessed 2D images. With respect to different types of surgical tools, different features may be relevant. In addition, depending on a particular surgical procedure, it may be known as to types of surgical tools that may be used during the surgery. As such, the image feature selector 320 may access surgical tool detection models 220 which may specify different types of features keyed on different types of surgical tools. Based on that configuration, the image feature selector 320 may determine, at 315, the types of features that may need to be extracted from 2D images in a particular surgery procedure. For instance, if a surgery is resection of liver, it may be known that the only surgical tools to be used are cutters and hooks. Given that, the image feature selector 320 may obtain information about the features to be extracted in order to recognize these two types of tools.

The features to be extracted are sent used to inform the image feature extractor 330. Upon receiving the instruction on what image features to be identified, the image feature extractor 330 extracts, at 325, such image features from the preprocessed images. The extracted features may then be used by the feature-based tool classifier 340 to classify, at 335, the tool type present in the image based on the extracted features. As image features of multiple types of surgical tools may need to be extracted from the same images, some features expected for a certain tool may not present solid characteristics of the surgical tool (e.g., features extracted for surgical cutter from images having surgical hook present therein). In this situation, the confidence of the classification for that type of tool may also be consistently low. In this situation, a classification result with a poor confidence, as determined at 345, may not be adopted and no classification is attained. The process continues to process the next images at 325. If the confidence score for a classification satisfies certain conditions, as determined at 345, the classification of the tool type is accepted. In this case, the surgical tool pose estimator 350 proceeds to estimate, at 355, the pose of the surgical tool. For example, if the detection identifies that the surgical tool observed in 2D images corresponds to a hook, the 3D location and orientation of the hook are determined. As discussed herein, such pose information is important in determining what is the attention point of the user so that to accurately determine the focused information to be displayed in a special way.

FIG. 3C shows a different exemplary realization of the image-based surgical tool classifier 300, in accordance with a different embodiment of the present teaching. In this illustrated embodiment, the image-based surgical tool classifier 300 performs classification via machine learned surgical tool classification models 360, which may be trained, based on training data, to recognize K different types of surgical tools. In some embodiments, the models 360 may have K outputs each of which corresponds to one type of surgical tool and is a numerical value between 0 and 1, representing, e.g., a probability that the input image includes a corresponding tool type. In this illustration, the surgical tool classification models 360 is trained to learn implicitly to perform the functions of image feature selection, extraction and then classification against all K types of tools based on the characteristics observed in the 2D images. With the output K probabilities, a model-based tool type determiner 370 may then select one of the classifications for K tool types as the most likely one based on the probabilities from the models 360. Such detected surgical tool type is then used by the surgical tool pose estimator 350 to analyze the images to determine the position and orientation of the detected tool. When the 2D images are registered with the 3D workspace coordinate system, the appearance of the detected surgical tool may be analyzed to derive the 3D location as well as the orientation of the tool in the 3D workspace coordinate system. As discussed above, both the 3D location and orientation of the tool, particularly the tip of the tool, are important in identifying the focused information.

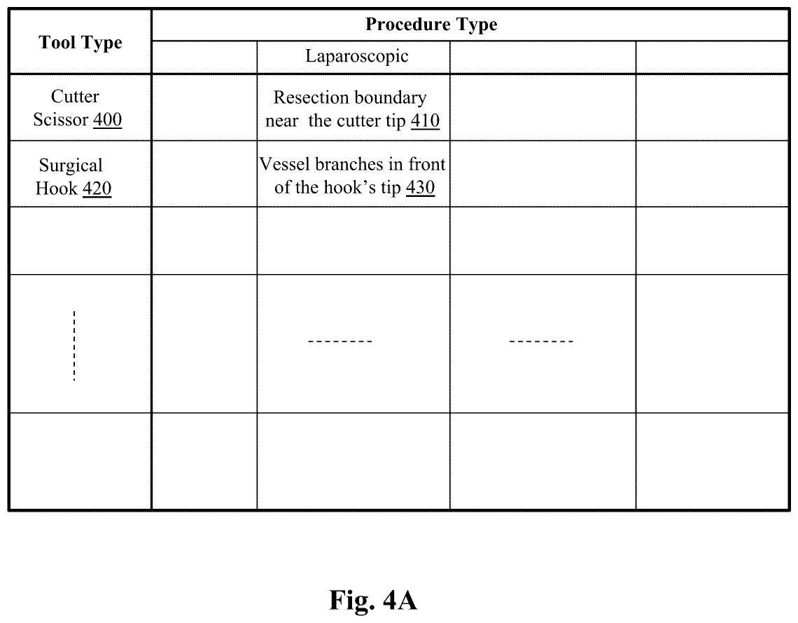

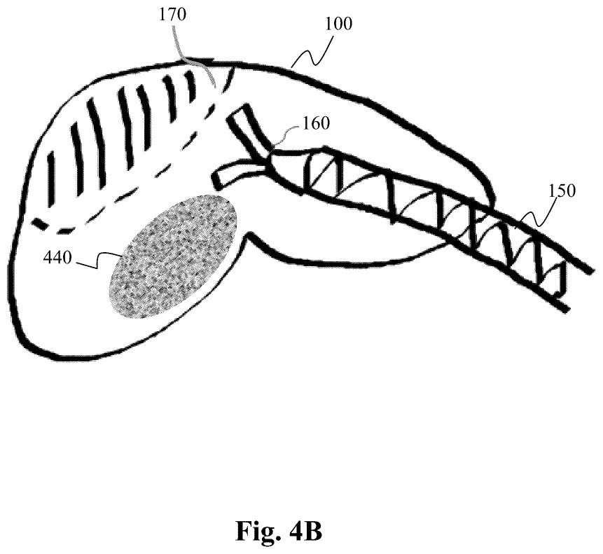

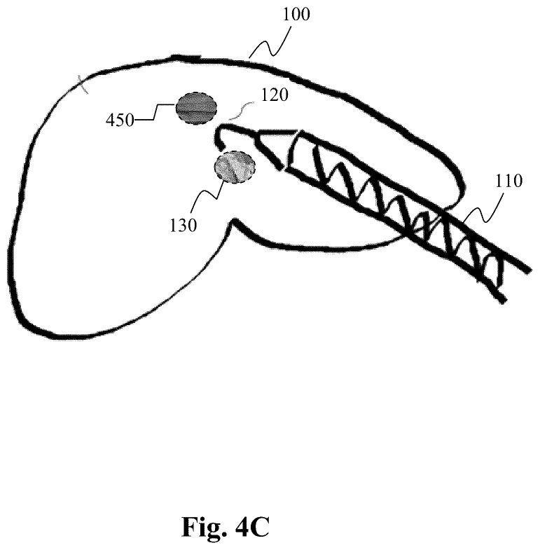

The output from the surgical tool detection unit 210 includes the surgical tool type as well as the pose of the tool tip and is sent to the tool-based focused region identifier 240, as shown in FIG. 2A for identifying the focused information based on the tool type and the focus area determined based on the pose of the tool tip. As disclosed previously, the type of focused information may be determined according to the tool/focused information configuration 250. Then based on the tip orientation and its spatial relationship with respect to the 3D model of the organ, the specific focused information may be identified. FIG. 4A shows an exemplary tool/focused information configuration as stored in 250, in accordance with an embodiment of the present teaching. In this example, there may be a table where in the rows may represent different tool types and columns may represent procedure types. The configuration may be structured this way because the same type of surgical tool may be used for different functions in different types of surgery procedures.

As illustrated in FIG. 4A, this exemplary configuration may include rows corresponding to different types of surgical tools such as cutter scissor 400, surgical hook 420, . . . , etc. Different columns correspond to different procedures so that each entry in the table corresponds to a particular combination of a surgical tool being used in a specific surgery procedure. Content provided in each entry may specify the type of focused information that a user (e.g., a surgeon) likely desires to see clearly. For instance, if a cutter scissor 400 is used in a laparoscopic procedure, the type of focused information is specified as “Resection boundary near the cutter tip” (410). FIG. 4B illustrates an example of resection boundary near the cutter tip displayed, in accordance with an embodiment of the present teaching. In this example, the cutter 160 is detected and its pose is determined. Based on the estimated cutter's pose, the spatial relationship between the cutter opening (tip) and the anatomy of the target organ may be determined via the 3D model of the organ in order to identify a specific portion of the resection boundary on the 3D model for special display. For example, the portion of the resection boundary to be used as focused information may correspond to the part of the resection boundary facing the opening of the cutter. Particularly, the resection boundary 170 nearing the cutter and facing the opening of the cutter is deemed as the focused information and displayed in a special way, even though organ information nearby the detected tool may also be displayed. For instance, the part of organ in 440 may also be display when the cutter is detected but may not be rendered in a special way because it is not facing the opening of the cutter 160.

As another example, if a surgical hook 420 is detected in 2D images during a laparoscopic procedure, the type of focused information is specified as “Vessel branches in front of the hook's tip” (430), as shown in FIG. 4A. In this case, because the type is a hook, the relevant information is vessel branches as configured. However, which part of the vessel tree corresponds to focused information may be determined based on the pose of the detected hook. This is illustrated in FIG. 4C, where a hook 120 is detected that has an orientation in such a way that the tip of the hook is in a certain direction as shown. In this case, the type of focused information is the vessel tree associated with the organ, but which part of the vessel tree is considered as focused information is determined based on the spatial relationship between the tip of the hook and the anatomy of the organ. In this example, the part of the vessel tree at, e.g., location 130 is facing directly with the tip of the hook and may be deemed as focused information. While the vessel branches in area 450 may not be considered as focused information even though it is also near the detected tool due to the fact that it is not in the direction of the tip of the hook.

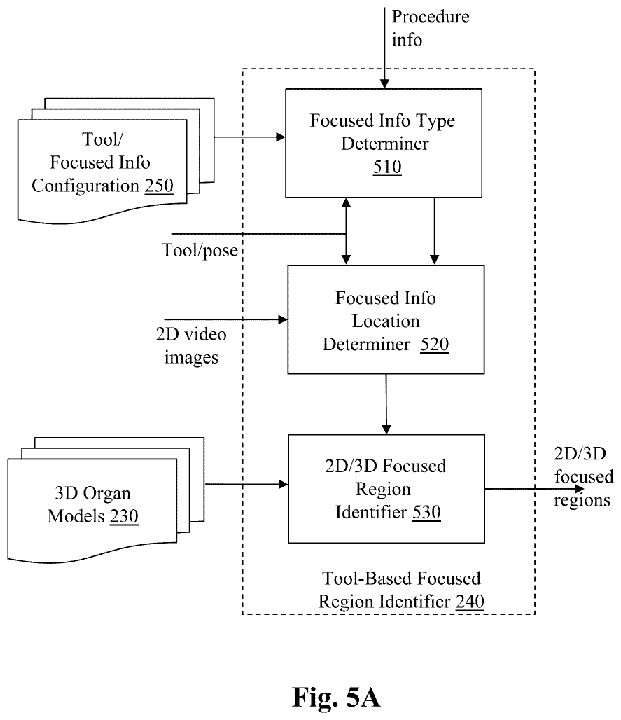

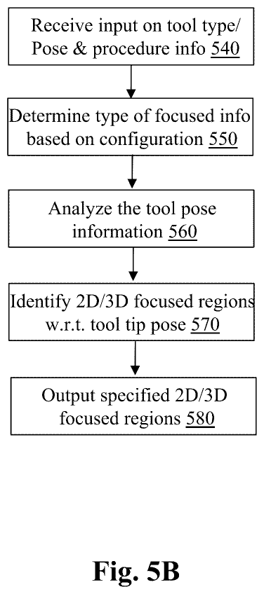

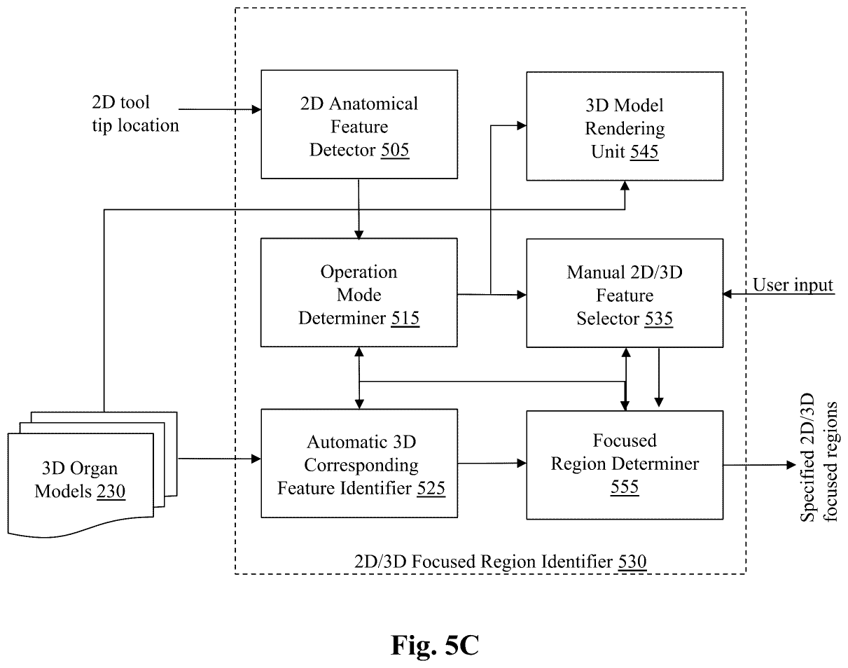

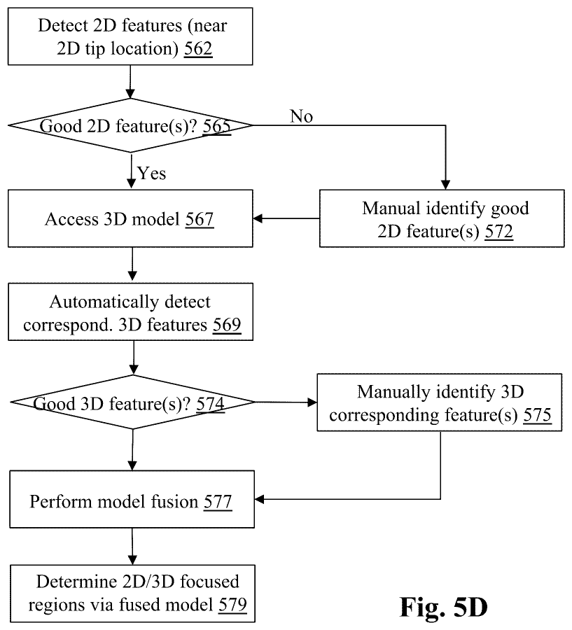

FIG. 5A depicts an exemplary high level system diagram of the tool-based focused region identifier 240, in accordance with an embodiment of the present teaching. In this illustrated embodiment, the tool-based focused region identifier 240 includes a focused information type determiner 510, a focused information location determiner 520, and 2D/3D focused region identifier 530. As discussed herein, to determine focused information, both specific information type and particular regions of interest (on both 2D images and 3D model) need to be identified. The focused information type determiner 510 is provided for determining the type of focused information based on, the configuration specified in the tool/focused information configuration 250. On the other hand, the focused information location determiner 520 is provided for identifying a 2D focused region of interest in 2D images where the type of interested focused information resides. As discussed herein, the 2D focused region is determined based on the pose (e.g., location and the direction) of the tip of the detected surgical tool. Based on the 2D focused region determined in 2D images, a 3D focused region corresponding to the 2D focused region may then be identified accordingly. This is achieved by the 2D/3D focused region identifier 530.

FIG. 5B is a flowchart of an exemplary process of the tool-based focused region identifier 240, in accordance with an embodiment of the present teaching. The focused information type determiner 510 receives, at 540, detection result on the surgical tool type and its pose and determines, at 550 based on the surgical tool type, the type (e.g., blood vessel) of focused information according to the configuration from 250 (illustrated in FIG. 4A). To determine the particular 2D region of interest where the type of focused information resides, the focused information location determiner 520 analyzes, at 560, the pose of the tool (e.g., the location and the orientation/direction of the tip of the tool). Such information is used by the 2D/3D focused region identifier 530 to identify, at 570, first a region of interest in 2D images, i.e., 2D focused region, with respect to the pose of the tool tip and correspondingly a part of the anatomic structure in the 3D model, i.e., a 3D focused region based on the 2D focused region. Such identified specific focused 2D and 3D focused regions may then be output at 580. As discussed herein, the 3D focused region is where the specific type of focused anatomic information (determined based on the type of surgical tool detected) resides so that the 3D information in the 3D focused region may be retrieved and projected onto the 2D focused region to assist the surgeon to operate using the surgical tool.

In some embodiments, other information in the 3D model near the detected tool location may also be retrieved and displayed to provide, e.g., better surgical context. For instance, when a hook is detected, the type of focused information may correspond to blood vessels. Although a specific portion of a vessel tree facing the tip of the tool may be deemed as specific focused information, 3D representation of other part of the vessel tree near the tool may also be output and displayed. In addition, the part of the organ around the vessels near the tool may also be output and displayed. The specific focused information may be displayed in a special way (e.g., with highlight or being colored or with a much higher contrast) and other related information around the special focused information may be displayed in a way that will not interfere or diminish the special effect of the special focused information.

FIG. 5C depicts an exemplary high level system diagram of the 2D/3D focused region identifier 530, in accordance with an embodiment of the present teaching. As discussed herein, the 2D and 3D focused regions may be identified from 2D images and 3D models, respectively. A 2D focused region may be determined based on an input location near a tip of the detected surgical instrument. This 2D focused region may have a corresponding 3D focused region. In some embodiments, to identify the 3D focused region corresponding to the 2D focused region, important anatomical features as observed in the 2D focused region in 2D images may be extracted and used to identify corresponding 3D anatomical features points in the 3D models. The 2D/3D focused region identifier 530 is provided to achieve that via either automated or semi-automated operations, as disclosed herein.

In this illustrated embodiment, the 2D/3D focused region identifier 530 may take a 2D instrument tip location as input and generate both 2D and 3D focused regions as output. To facilitate the operation, the 2D/3D focused region identifier 530 comprises a 2D anatomical feature detector 505, an operation model determiner 515, an automatic 3D corresponding feature identifier 525, a manual 2D/3D feature selector 535, a 3D model rendering unit 545, and a focused region determiner 555. FIG. 5D is a flowchart of an exemplary process of the 2D/3D focused region identifier 530, in accordance with an embodiment of the present teaching. In operation, when the 2D anatomical feature detector 505 receives the 2D instrument tip location as an input, it may first align, via anatomical feature points, the 3D models with the 2d images. To do so, it may automatically detect 2D features at 562. In some embodiments, such 2D feature points may be obtained near or around the input tip location. In some situations, such 2D features may correspond to some anatomically distinct features, e.g., a concave point between two halves of a liver (as shown in FIGS. 1A-1B, and 4B-4C) or some fork point between two blood vessel branches. The type of such feature points may also be dependent on the instrument type detected. For instance, if the detected instrument or tool is a surgical hook, it may indicate that the surgeon intends to do something with the blood vessels near the tip of the tool. In this situation, a branching point in a blood vessel tree may represent better features than the concave point between different parts of a liver. It is also so when such distinct features may facilitate the identification of the corresponding 3D features from a part of the 3D models for 3D representation of the blood vessels.

In some situations, the input tip location may be such that there is no such distinct feature present. For instance, the instrument tip may be near the surface of a liver. In addition, in some situations, the detected 2D features may not be as good as needed. In the event that no adequately good 2D feature is detected via automatic means, the present teaching enables quality 2D feature detection by resorting to human assistance. The operation model determiner 515 may assess, at 565, whether the automatically detected 2D features possess a desirable level of distinctiveness or quality for being used to identify corresponding 3D features. If so, an automatic operation mode is applied and the automatic 3D corresponding feature identifier 525 is activated, which then accesses, at 567, the 3D models 230 and automatically detect, at 569, 3D anatomical features from the 3D models that correspond to the detected 2D anatomical features.

If the 2D anatomical features are not satisfactory (i.e., either not detected or not of a good quality), determined at 565, the operation model determiner 515 may control the process to identify 2D features in a manual mode by activating the manual 2D/3D feature selector 535, which may control an interface to communicate with a user to facilitate the user to manually identify, at 572, the 2D features. Such manually identified 2D anatomical features are then provided to the automatic 3D corresponding feature identifier 525 for accessing, at 567, the 3D models and then automatically detect, at 569, from the 3D models, the 3D anatomical features corresponding to the 2D features. To ensure quality of identified 3D corresponding anatomical features, the automatic 3D corresponding feature identifier 525 may assess, at 574, whether the identified 3D features are satisfactory based on some criteria. If they are satisfactory, both the 2D features and the corresponding 3D features may be fused in order to identify the 2D and 3D focused regions.

In the event that the 3D anatomical features are not satisfactory, the operation mode determiner 515 may activate the 3D model rendering unit 545 to render the 3D models in order to facilitate the manual 2D/3D feature selector 535 to interact with a user to manually identify, at 575, 3D corresponding anatomical features from the rendered 3D models. With the satisfactory corresponding 3D anatomical features (either automatically identified or manually selected), the 2D features and the corresponding 3D features can now be used to align (or orient) the 3D models with what is observed in 2D images (consistent with the perspective of the camera). That is, the 2D information as observed in 2D images (represented by, e.g., the 2D anatomical features) is fused, at 577, with 3D information from the 3D models (represented by, e.g., the corresponding 3D anatomical features) so that the focused region determiner 555 may then proceed to accordingly determine, at 579, the 2D focused region and the corresponding 3D focused region based on the fused 2D/3D information. As seen in FIG. 5A, the 2D and 3D focused regions may then be used by the focused information display unit 260 for displaying tool-relevant information to a user to facilitate the surgical operation.

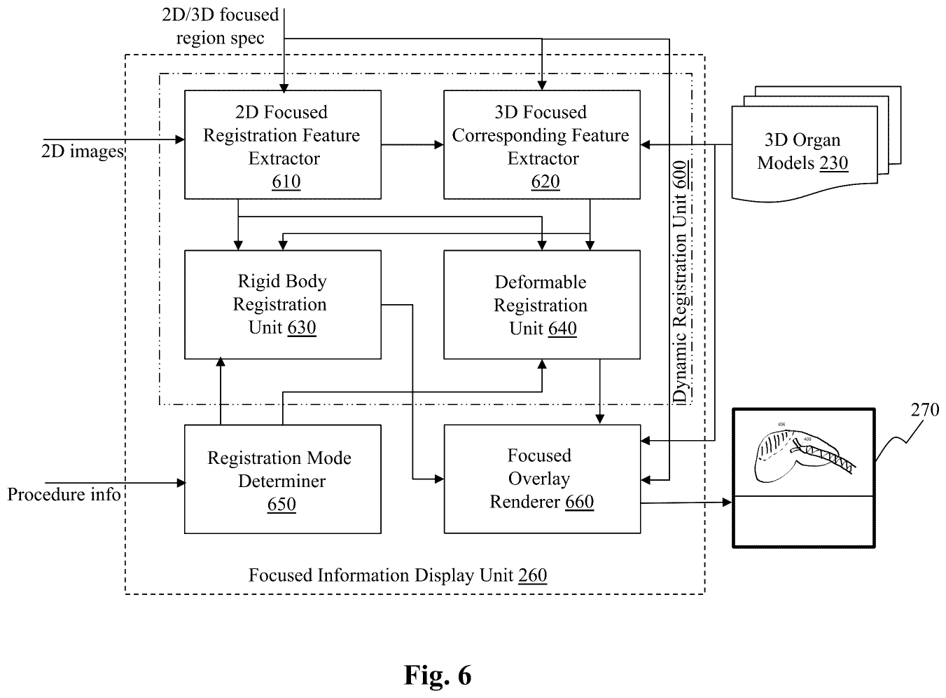

FIG. 6 is an exemplary high level system diagram of the focused information display unit 260, in accordance with an embodiment of the present teaching. As disclosed herein, the function of the focused information display unit 260 is to visualize specific information dynamically determined based on the currently deployed surgical tools. These visualizations may then assist a user to perform a specific procedure. The display is accomplished near the tool tip as can be observed from the perspective of the tip of the tool. The focused information identified in such a manner is intended to be consistent with what the tool is to be used for at the moment so that the focused information may be rendered to better facilitate the user to perform a specific operation.

In this illustrated embodiment, the focused information display unit 260 comprises a registration mode determiner 650, a dynamic registration unit 600, and a focused overlay renderer 660. The registration mode determiner 650 may be provided to determine the mode of registration before rendering the 3D model information representing the 3D focused information onto the 2D images. In some situations, the registration mode may be for registering a rigid body (e.g., when the organ being operated on is mostly rigid such as bones). In other situations, the registration mode may have to be directed to registration of deformable object such as a heart in an operation where the heart may deform over time due to, e.g., pumping the blood or patient's breathing. The dynamic registration unit 600 is provided for registering the 3D information to be rendered with the 2D images (including, e.g., both focused and non-focused information) in a registration mode determined by the registration mode determiner 650. The focused overlay renderer 660 is provided to render the 3D focused information and non-focused information in 2D images based on registration result.

In some embodiments, the dynamic registration unit 600 may further comprise a 2D focused registration feature extractor 610 for extracting 2D feature points to be used for registration, a 3D focused corresponding feature extractor 620 for identifying 3D feature points corresponding to the 2D feature points, a rigid body registration unit 630 provided for performing rigid registration if it is called for, and a deformable registration unit 640 for performing deformable registration in a deformable registration mode. The registration result from either rigid registration or deformable registration is sent to the focused overlay renderer 660 so that the focused and non-focused information from the 3D model may be rendered by appropriately projecting the 3D information onto the 2D images based on the registration result.

In some embodiments, the dynamic registration unit 600 may also be realized using a model based registration implementation wherein deep-learned models (not shown) may be obtained via training data so that feature extraction and registration steps performed explicitly by 2D focused registration feature extractor 610, 3D focused corresponding feature extractor 620, rigid body registration unit 630, and deformable registration unit 640, may be instead performed implicitly via deep-learned models that incorporate knowledge learned in training in their embeddings and parameters of other layers. Such a model-based solution may take 2D images, 3D models and identified 2D/3D focused regions of interest as input and generate a registration result as output to be provided to the focused overlay renderer 660.

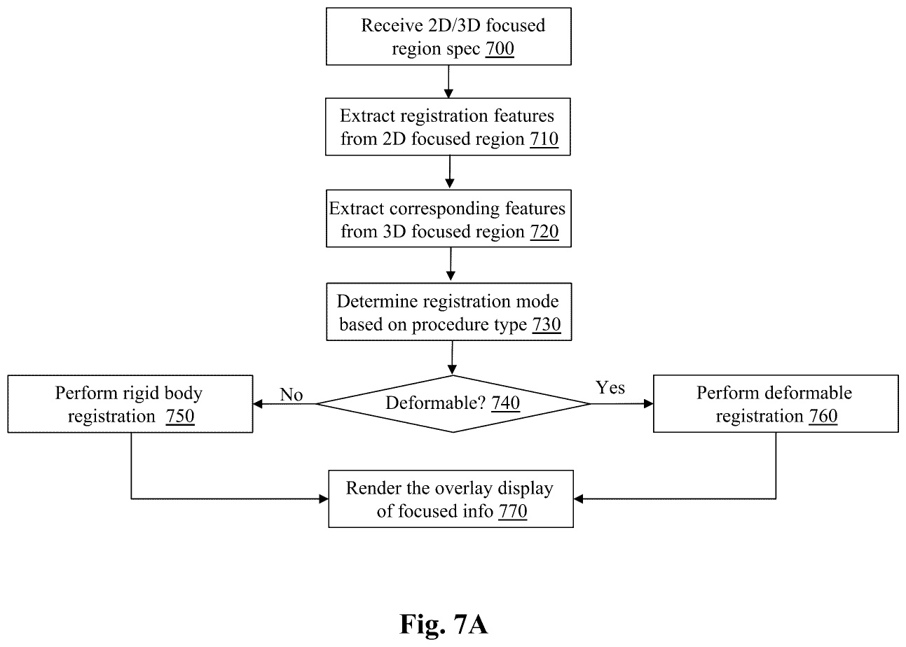

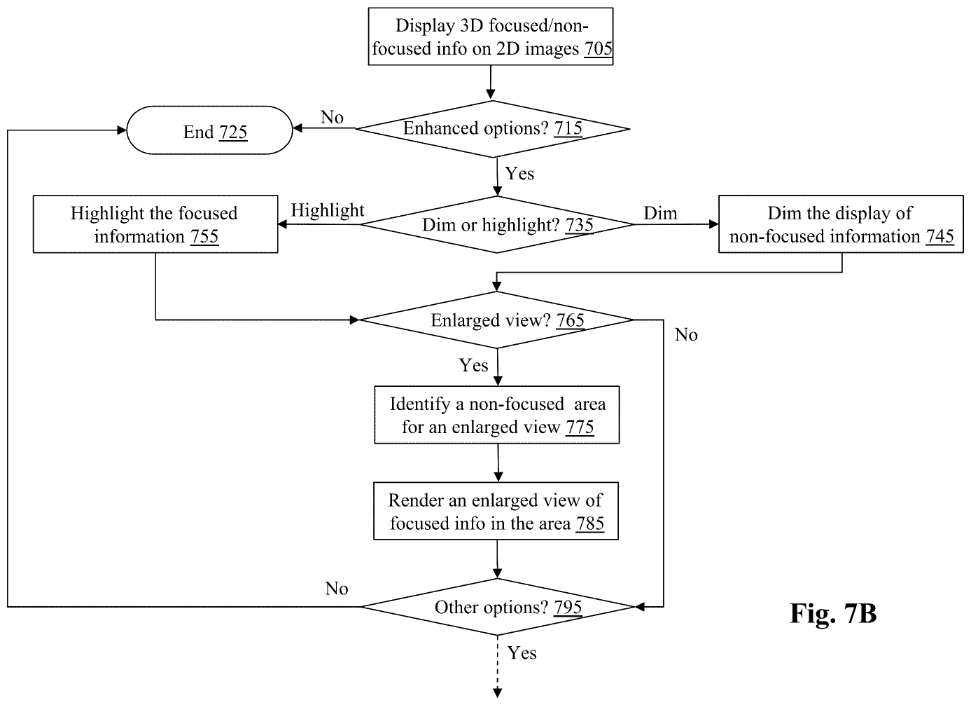

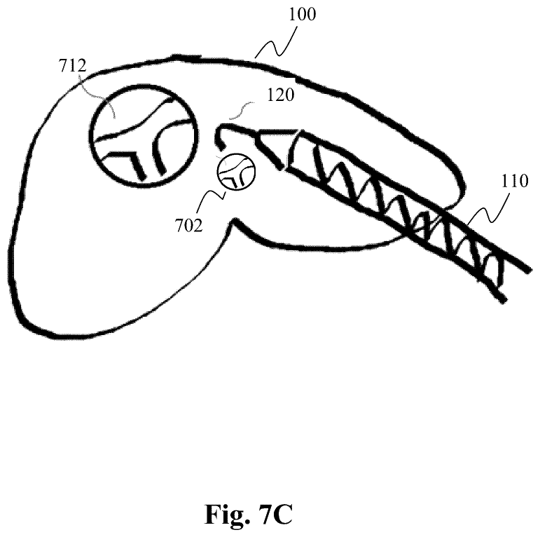

FIG. 7A is a flowchart of an exemplary process of the focused information display unit 260, in accordance with an embodiment of the present teaching. When 2D focused registration feature extractor 610 receives, at 700, specified 2D/3D focused regions, it extracts, at 710, registration feature points from 2D images. In some embodiments, the feature points may be extracted from the specified focused regions. In some embodiments, the feature points to be used for registration may be extracted from anywhere on the target organ, regardless such feature points are from the focused region of interests or not. In some embodiments, the 2D feature points for registration may be identified manually by a user through interactions with the 2D images. With the identified 2D feature points, the 3D focused corresponding feature extractor 620 may proceed to extract, at 720, 3D feature points from the 3D model 230 for the target organ that correspond to the 2D feature points.

In the meantime, registration mode determiner 650 may determine, at 730, the registration mode to be applied based on the type of the surgical procedure at issue. As discussed herein, in some situations, rigid registration may be performed if the involved anatomical structures are rigid bodies (e.g., bones) but in some situations, deformable registration may be used when the anatomical parts involved do deform over time. When a rigid registration is applicable, determined at 740, the rigid body registration unit 630 performs, at 750, the registration based on 2D feature points as well as their corresponding 3D feature points. When a deformable registration is applied, the deformable registration unit 640 performs, at 760, deformable registration, at 760, based on the 2D and 3D corresponding feature points. Upon completion of registration, the focused overlay renderer 660 renders, at 770, the focused information by projecting, based on the registration result, the 3D focused information from the 3D model onto the 2D images. In presenting information around the detected tool, other information, although not focused information but nevertheless in the vicinity of the surgical tool may also be rendered. As discussed herein, the focused and non-focused information may be rendered in different ways so that the focused information is display in a special way to create a clearer visual to the user while the non-focused information may be displayed in a way that provides the context without taking away the attention from the focused information.

As discussed herein, there are different ways to render the focused information. Any implementation can be adopted so long as the focused information provides a clear visual to a user in contrast with the surrounding non-focused information. In this disclosure, some exemplary ways to render the focused information are provided but they are merely for illustration instead of as limitations. FIG. 7B is a flowchart of an exemplary process of the focused overlay renderer 660 for rendering focused information identified based on a tool detected, in accordance with an embodiment of the present teaching. When the focused overlay renderer 660 receives the registration result and the 2D/3D focused region specification, it retrieves relevant information from the 3D models 230 and displays, at 705, the 3D focused and non-focused information by projecting such information onto 2D images in accordance with the registration result.

For example, when a detected surgical tool is a cutter scissor and the surgery is on liver resection in a laparoscopic procedure, the identified focused information may be a portion of the surface of the liver with a section of the resection boundary characterized by the 3D model for the target liver. The section of the resection boundary identified as focused information may be a part of a resection trajectory that is faced with the opening of the cutter, determined based on the orientation of the cutter. In this example, the non-focused information may include a portion of the surface of the liver as modeled where the portion of the liver surface is similarly determined as near the opening location or orientation of the cutter detected from 2D images. With both focused and non-focused information determined, their 3D representations from the 3D model are projected onto the 2D images. For example, the portion of the surface of the liver (non-focused but relevant information that provides the visual context for the focused information) is rendered with the part of resection boundary (focused information) on the surgical trajectory superimposed as, e.g., cutting points at appropriate positions on the surface.

In some embodiments, focused and non-focused information may be rendered in special ways to enhance the quality of visual assistance to a user during a surgery. As discussed herein, some enhanced display options may be provided to improve the quality of the visual guidance. If the focused overlay renderer 660 does not support such enhanced display options, determined at 715, its display operation ends at 725. Otherwise, the focused overlay renderer 660 may be configured, by a user in each surgery, to apply certain enhanced display options based on the needs of the user. In this illustration, for example, the enhanced display process may check, at 735, whether it is configured to either dim the non-focused information or highlight the focused information displayed. If it is configured to dim the non-focused information (or even other background information in the 2D images), the focused overlay renderer 660 dims, at 745, the displayed non-focused information (and other parts of the 2D images). If it is configured to highlight the focused information, the focused overlay renderer 660 modifies the display of the focused information at 755. The modification may be applied to make the focused information visually more pronounced. Using the example above with focused information being a section of the resection boundary, the cut points of the section projected on the 2D images may be rendered using a bright color or with a maximum intensity. In some embodiments, both dim and highlight may be applied (not shown in FIG. 7B) to further increase the contrast between focused information and other information in the 2D images.

Another exemplary enhanced display option may be to provide an enlarged view of the part of the 2D image where the focused information is rendered. If the focused overlay renderer 660 is configured to do so, determined at 765, an area on the display screen may be identified, at 775, for presenting an enlarged view of a region of interest in the 2D image where the focused information is rendered. Then an enlarged view of the content in the region of interest may be generated and displayed, at 785, in the area of the display screen for the enlarged view. FIG. 7C illustrates an exemplary display of focused information on blood vessels 702 next to the tip a surgical hook 120 with an enlarged view 712 of 702, in accordance with an embodiment of the present teaching. As shown, the enlarged view 712, although containing the same information as what is in 702, provides a much better visualization to a user and thus offers improved guidance to a user during the surgery. There may be other enhanced display options. If so, determined at 795, the focused overlay renderer 660 continues the process of generating improved visualization as to focused information. If there are no other options, the process moves to end the process at 725.

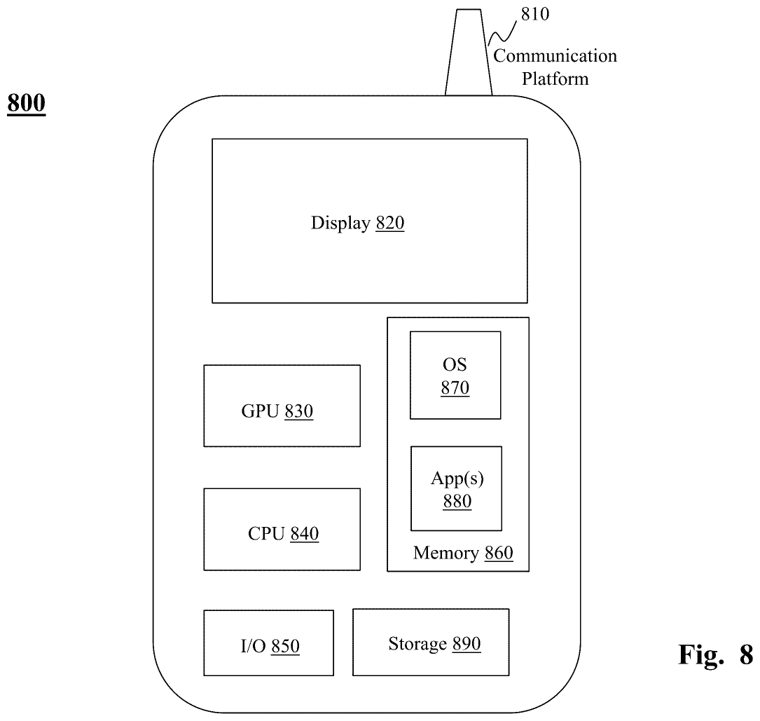

FIG. 8 is an illustrative diagram of an exemplary mobile device architecture that may be used to realize a specialized system implementing the present teaching in accordance with various embodiments. In this example, the user device on which the present teaching may be implemented corresponds to a mobile device 800, including, but not limited to, a smart phone, a tablet, a music player, a handled gaming console, a global positioning system (GPS) receiver, and a wearable computing device, or in any other form factor. Mobile device 800 may include one or more central processing units (“CPUs”) 840, one or more graphic processing units (“GPUs”) 830, a display 820, a memory 860, a communication platform 810, such as a wireless communication module, storage 890, and one or more input/output (I/O) devices 850. Any other suitable component, including but not limited to a system bus or a controller (not shown), may also be included in the mobile device 800. As shown in FIG. 8, a mobile operating system 870 (e.g., iOS, Android, Windows Phone, etc.), and one or more applications 880 may be loaded into memory 860 from storage 890 in order to be executed by the CPU 840. The applications 880 may include a user interface or any other suitable mobile apps for information analytics and management according to the present teaching on, at least partially, the mobile device 800. User interactions, if any, may be achieved via the I/O devices 850 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result the drawings should be self-explanatory.

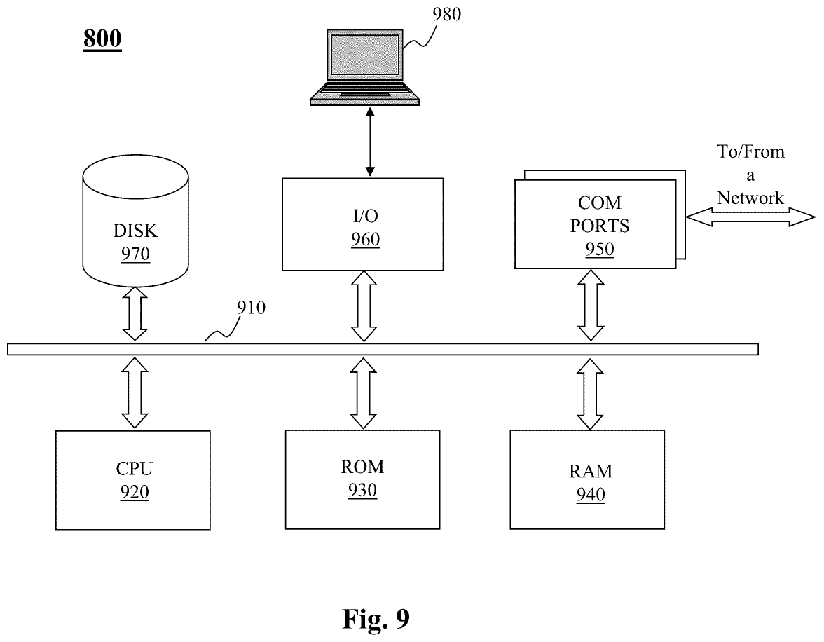

FIG. 9 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special purpose computer. Both can be used to implement a specialized system for the present teaching. This computer 800 may be used to implement any component or aspect of the framework as disclosed herein. For example, the information analytical and management method and system as disclosed herein may be implemented on a computer such as computer 900, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 900, for example, includes COM ports 950 connected to and from a network connected thereto to facilitate data communications. Computer 900 also includes a central processing unit (CPU) 920, in the form of one or more processors, for executing program instructions. The exemplary computer platform includes an internal communication bus 910, program storage and data storage of different forms (e.g., disk 970, read only memory (ROM) 930, or random-access memory (RAM) 940), for various data files to be processed and/or communicated by computer 900, as well as possibly program instructions to be executed by CPU 920. Computer 900 also includes an I/O component 960, supporting input/output flows between the computer and other components therein such as user interface elements 980. Computer 900 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method implemented on at least one processor, a memory, and a communication platform, comprising:
receiving two-dimensional (2D) images capturing anatomical structures and a surgical instrument present in a surgery;
detecting a tool attached to the surgical instrument from the 2D images, wherein the tool is of a type with a pose, including a location and an orientation;
determining focused information in the 2D images based on the type and the pose of the tool, wherein the focused information is for generating a visual guide to assist a user in performing a surgical task using the tool; and
generating, via model fusion, the visual guide based on the focused information.

2. The method of claim 1, wherein the step of detecting comprises:
extracting a plurality of features from the 2D images, wherein the features relate to types of tools that can be deployed in the surgery;
classifying, based on the plurality of features, against the types of tools;
identifying the type of the tool based on the classification result; and
determining, based on 2D images, the location, and the orientation of the tool.

3. The method of claim 1, wherein the step of detecting comprises:
generating, based on the 2D images using machine learned surgical tool classification models, classification results with respect to a plurality of types of surgical tools, wherein the machine learned surgical tool classification models are obtained via training on the plurality of types of surgical tools;
determining the type of the tool captured in the 2D images based on the classification results; and
determining, based on 2D images, the location, and the orientation of the tool.

4. The method of claim 1, wherein the surgical task is estimated based on the type of tool detected and the surgery.

5. The method of claim 1, wherein the step of selecting the focused information comprises:
accessing a configuration specifying one or more types of focused information for a combination of a surgical procedure and a surgical tool used in the surgical procedure;
identifying the focused information type specified in the configuration with respect to a combination of the surgery and the type of tool detected from the 2D images;
determining the focused information of the focused information type from a region of the 2D images, wherein the region is determined based on the pose of the tool; and
outputting the determined focused information.

6. The method of claim 1, wherein the step of generating the visual guide comprises:
registering a 3D model modeling the anatomical structures with the 2D images;
retrieving 3D information from the 3D model, wherein the 3D information corresponds to visual information in proximity of the tool in the 2D images;
identifying, from the 3D information, 3D focused information corresponding to the focused information and/or 3D non-focused information surrounding of the focused information;
creating the visual guide by projecting the 3D focused information and/or the 3D non-focused information onto the 2D images based on the registering result.

7. The method of claim 6, further comprising enhancing the visual guide with at least one of:
dimming the presentation of the projected 3D non-focused information;
highlighting the presentation of the projected 3D focused information; and
creating an additional enlarged view of a sub-region in the 2D images where the 3D focused information is projected.

8. Machine readable and non-transitory medium having information recorded thereon, wherein the information, when read by the machine, causes the machine to perform the following steps:
receiving two-dimensional (2D) images capturing anatomical structures and a surgical instrument present in a surgery;
detecting a tool attached to the surgical instrument from the 2D images, wherein the tool is of a type with a pose, including a location and an orientation;
determining focused information in the 2D images based on the type and the pose of the tool, wherein the focused information is for generating a visual guide to assist a user in performing a surgical task using the tool; and
generating, via model fusion, the visual guide based on the focused information.

9. The medium of claim 8, wherein the step of detecting comprises:
extracting a plurality of features from the 2D images, wherein the features relate to types of tools that can be deployed in the surgery;
classifying, based on the plurality of features, against the types of tools;
identifying the type of the tool based on the classification result; and
determining, based on 2D images, the location, and the orientation of the tool.

10. The medium of claim 8, wherein the step of detecting comprises:
generating, based on the 2D images using machine learned surgical tool classification models, classification results with respect to a plurality of types of surgical tools, wherein the machine learned surgical tool classification models are obtained via training on the plurality of types of surgical tools;
determining the type of the tool captured in the 2D images based on the classification results; and
determining, based on 2D images, the location, and the orientation of the tool.

11. The medium of claim 8, wherein the surgical task is estimated based on the type of tool detected and the surgery.

12. The medium of claim 8, wherein the step of selecting the focused information comprises:
accessing a configuration specifying one or more types of focused information for a combination of a surgical procedure and a surgical tool used in the surgical procedure;
identifying the focused information type specified in the configuration with respect to a combination of the surgery and the type of tool detected from the 2D images;
determining the focused information of the focused information type from a region of the 2D images, wherein the region is determined based on the pose of the tool; and
outputting the determined focused information.

13. The medium of claim 8, wherein the step of generating the visual guide comprises:
registering a 3D model modeling the anatomical structures with the 2D images;
retrieving 3D information from the 3D model, wherein the 3D information corresponds to visual information in proximity of the tool in the 2D images;
identifying, from the 3D information, 3D focused information corresponding to the focused information and/or 3D non-focused information surrounding of the focused information;
creating the visual guide by projecting the 3D focused information and/or the 3D non-focused information onto the 2D images based on the registering result.

14. The medium of claim 13, wherein the information, when read by the machine, further causes the machine to perform the step of enhancing the visual guide with at least one of:
dimming the presentation of the projected 3D non-focused information;
highlighting the presentation of the projected 3D focused information; and
creating an additional enlarged view of a sub-region in the 2D images where the 3D focused information is projected.

15. A system, comprising:
a surgical tool detection unit implemented by a processor and configured for
receiving two-dimensional (2D) images capturing anatomical structures and a surgical instrument present in a surgery, and
detecting a tool attached to the surgical instrument from the 2D images, wherein the tool is of a type with a pose, including a location and an orientation;
a tool-based focused information identifier implemented by a processor and configured for determining focused information in the 2D images based on the type and the pose of the tool, wherein the focused information is for generating a visual guide to assist a user in performing a surgical task using the tool; and
a focused information display unit implemented by a processor and configured for generating, via model fusion, the visual guide based on the focused information.

16. The system of claim 15, wherein the surgical tool detection unit comprises:
an image feature extractor implemented by a processor and configured for extracting a plurality of features from the 2D images, wherein the features relate to types of tools that can be deployed in the surgery;
a feature-based tool classifier implemented by a processor and configured for
classifying, based on the plurality of features, against the types of tools, and
identifying the type of the tool based on the classification result; and
a surgical tool pose estimator implemented by a processor and configured for determining, based on the 2D images, the location, and the orientation of the tool.

17. The system of claim 15, wherein the surgical tool detection unit comprises:
machine learned surgical tool classification models implemented by a processor, obtained via training on a plurality of types of surgical tools, and configured for
generating, based on the 2D images, classification results with respect to the plurality of types of surgical tools, and
a model-based tool type determiner implemented by a processor and configured for determining the type of the tool captured in the 2D images based on the classification results; and
a surgical tool pose estimator implemented by a processor and configured for determining, based on the 2D images, the location, and the orientation of the tool.

18. The system of claim 15, wherein the surgical task is estimated based on the type of tool detected and the surgery.

19. The system of claim 15, wherein the tool-based focused information identifier comprises:
a focused information type determiner implemented by a processor and configured for accessing a configuration specifying one or more types of focused information for a combination of a surgical procedure and a surgical tool used in the surgical procedure;
a focused information location determiner implemented by a processor and configured for:
identifying the focused information type specified in the configuration with respect to a combination of the surgery and the type of tool detected from the 2D images,
determining the focused information of the focused information type from a region of the 2D images, wherein the region is determined based on the pose of the tool, and
outputting the determined focused information.

20. The system of claim 15, wherein the focused information display unit comprises:
a dynamic registration unit implemented by a processor and configured for registering a 3D model modeling the anatomical structures with the 2D images; and
a focused overlay renderer implemented by a processor and configured for
retrieving 3D information from the 3D model, wherein the 3D information corresponds to visual information in proximity of the tool in the 2D images,
identifying, from the 3D information, 3D focused information corresponding to the focused information and/or 3D non-focused information surrounding of the focused information, and
creating the visual guide by projecting the 3D focused information and/or the 3D non-focused information onto the 2D images based on the registering result.

21. The system of claim 20, wherein the focused overlay renderer is further configured for enhancing the visual guide by performing at least one of:
dimming the presentation of the projected 3D non-focused information;
highlighting the presentation of the projected 3D focused information; and
creating an additional enlarged view of a sub-region in the 2D images where the 3D focused information is projected.
